(19) United States
(12) Patent Application Publication
(10) Pub. No.: US 2023/0293114 A1
(43) Pub. Date: Sep. 21, 2023

(54) Electronic device for monitoring a neurophysiological state of an operator in an aircraft control station, associated monitoring method and associated computer program

(71) Applicant: THALES, COURBEVOIE (FR)

(72) Inventor: Bastien BERTHELOT, TOULOUSE (FR)

(21) Appl. No.: 18/183,940

(22) Filed: Mar. 14, 2023

(30) Foreign Application Priority Data
Mar. 15, 2022 (FR) 2202250

(51) Int. Cl. A61B 5/00; A61B 5/024; A61B 5/318; A61B 5/369

(52) U.S. Cl. CPC A61B 5/7264; A61B 5/02416; A61B 5/318; A61B 5/746; A61B 5/369

(57) ABSTRACT

The invention relates to an electronic device for monitoring a neurophysiological state of an operator in a control station of an aircraft including a receiver module configured for receiving a datum from a sensor, a categorization module configured for associating, from the data received, a category with the operator, a processing module configured for extracting from each datum, at least one parameter representative of the neurophysiological state of the operator, and a detection module configured for applying a model derived from a machine learning method, for determining, according to the representative parameters, whether the operator is in a nominal neurophysiological state or in an altered neurophysiological state, the model being chosen from a list of predetermined models according to the category associated with the operator.

### REFERENCE TO RELATED APPLICATION

This application is a U.S. non-provisional application claiming the benefit of French Application No. 22 022250, filed on Mar. 15, 2022, the contents of which are incorporated herein by reference in their entirety.

### TECHNICAL FIELD OF THE INVENTION

The present invention relates to an electronic device for monitoring a neurophysiological state of an operator in an aircraft.

The invention further relates to a method for controlling the monitoring of a neurophysiological state of an operator in an aircraft.

The invention further relates to a computer program including software instructions which, when executed by a computer, implement such a method.

### BACKGROUND OF THE INVENTION

The aircraft is typically an airplane, a helicopter, or a drone. The operator is, e.g., the pilot of the aircraft, the co-pilot, or a radar operator. The control station is in particular arranged in the aircraft or is arranged at a distance from the aircraft in the case, e.g., of a drone.

Monitoring the neurophysiological state of such an operator is essential for the safety of the aircraft in order to detect any deleterious state: fatigue, mental load, incapacitation, stress, etc., which could result in the operator's incapacity or deterioration of ability to perform the tasks expected under operational flight conditions.

Conventionally, the neurophysiological state of an operator is monitored by other aircraft operators. The co-pilot, e.g., monitors the neurophysiological state of the pilot, and vice versa.

To complement such monitoring, it has been proposed to monitor by means of one or a plurality of sensors arranged in the aircraft. The sensors measure a certain datum and the neurophysiological state of the operator is deduced therefrom.

However, such a method is not entirely satisfactory. Indeed, the method is not sufficiently robust. In particular, the monitoring method needs to cope with the variabilities of the situations encountered. Indeed, the detection of an altered neurophysiological state has to cope with the variability of the physiological characteristics of the different operators (age, gender, etc.), and also the variability of the environment wherein the operator is located (turbulent aircraft cockpit, cockpit where ambient luminosity varies, etc.).

Furthermore, the method has to be robust despite the few operational data available corresponding to the behaviors sought. In the military field, such problem is all the more complex because the data collected and useful for the application of interest are not necessarily available since the data are often classified and few because of the difficulty of observing critical situations.

To meet such requirement, methods have been proposed for monitoring only a particular mental state of the operator such as fatigue or mental load, etc. The method is then trained on a limited amount of data. Although the monitoring may be satisfactory during training of the algorithm, the generalization of the model, i.e., the adaptation thereof to a new unknown subject, often leads to a sharp drop in performance. Such a method is very specific to the data on which the training is carried out and thus, again, does not sufficiently cope with the variabilities subsequently encountered.

On the other hand, other methods have been proposed where algorithms are trained on large amounts of data, covering a large number of variabilities, but the algorithms then offer lower performance, and are insufficient for aeronautical applications and the high safety constraints which apply in the field of aeronautics.

In general, from conventional methods and from the bibliography, there is a clear need to make a choice between the performance of the method and the coverage thereof, i.e., the quantity and variability of the subjects on which the algorithm of the method was trained.

Finally, it should be noted that it is necessary that the method can be implemented in an aircraft and thus with limited on-board calculation resources, while determining the neurophysiological state of the operator in almost “real-time”. Detecting loss of consciousness, e.g., of a pilot during take-off has to be possible in less than a few seconds, typically in less than two seconds.

### SUMMARY OF THE INVENTION

An objective of the invention is to provide an electronic device for monitoring a neurophysiological state of an operator in an aircraft, for overcoming the difficulties explained hereinabove, in particular by offering precise, reliable and monitoring coping with the variabilities of the monitored operator.

To this end, the subject matter of the invention is an electronic device for monitoring a neurophysiological state of an operator in an aircraft, the monitoring device including:
a receiver module configured for receiving data from at least one sensor on-board the aircraft, each sensor being configured for measuring at least one piece of information relating to the operator;
a categorization module configured for associating the operator with one category from a list of predetermined categories, from the received datum or data;
a processing module configured for extracting from each datum, at least one parameter representative of the neurophysiological state of the operator; and
a detection module configured for receiving the category associated with the operator and the representative parameter(s), the detection module being further configured for applying a model derived from a machine learning method, for determining, according to the representative parameters, whether the operator is in a nominal neurophysiological state or in an altered neurophysiological state, the model being chosen from a list of predetermined models according to the category associated with the operator.

Thus, the present invention may be used for addressing the problem of the compromise between performance and coverage of monitoring. Indeed, prior categorization of the operator ensures, despite the possible combinations of variabilities, sufficient coverage of the monitoring, while offering higher performance on each specific model. Overall, by averaging the performance of models specific to each operator category, a higher detection performance is obtained than with a unique model which would seek to cover all operator variabilities. Indeed, the operator is monitored using a specific model for each category, while ensuring the coverage of all combinations by separation into different categories.

According to other advantageous aspects of the invention, the monitoring electronic device includes one or a plurality of the following features, taken individually or according to all technically possible combinations:

the monitoring device further includes a warning module configured for issuing a warning signal when the detection module determines that the operator is in an altered neurophysiological state;

each sensor is chosen from the group consisting of:
a cardiac sensor, in particular an electrocardiograph;
a pulse oximeter, in particular a photoplethysmography sensor;
a respiration sensor;
an accelerometer;
a scalp electrode, e.g., an electroencephalograph;
a pressure sensor arranged in an operator's seat;
a pressure sensor arranged in a control device suitable for being actuated by the operator;
a sweating sensor for the operator;
a galvanic skin response sensor;
a camera configured for taking at least one image including at least part of the operator;
a microphone;
an infrared sensor for the operator's skin temperature;
an internal temperature sensor for the operator; and
a near-infrared spectroscopy headband.

the categorization module is configured for associating a category according to at least one so-called individual attribute chosen from the group consisting of: gender, age, ethnicity, pilosity, hair length, presence of elements on the skin of the face;

the categorization module is configured for associating a category according to at least one attribute called a worn accessory chosen from the group consisting of: wearing glasses, polarized or non-polarized glasses, lenses, surgical mask, gas mask, headphone, cap;

the processing module is configured for extracting from each datum, at least one parameter representative of the neurophysiological state of the operator according to the category associated with the operator; and

the processing module is configured for extracting from each datum, at least one parameter representative of the neurophysiological state of the operator by implementing, for each datum, an algorithm chosen from the group consisting of:
an extraction of a predetermined characteristic of the associated datum followed by a machine learning method;
a deep learning method applied directly to the associated datum; and
a predetermined modeling applied to the associated datum.

the algorithm used is chosen according to the category of the operator.

The invention further relates to a method for monitoring a neurophysiological state of an operator in an aircraft control station, the method including at least:

receiving data from at least one sensor on-board the aircraft, each sensor being configured for measure at least one piece of information; relating to the operator;

associating with the operator one category from a list of predetermined categories from the received datum or data;

extracting from each datum at least one parameter representative of the neurophysiological state of the operator; and

receiving the category associated with the operator and the representative parameter(s) and application of a model derived from a machine learning method, for determining, according to the representative parameters, whether the operator is in a nominal neurophysiological state or in an altered neurophysiological state, the model being chosen from a list of predetermined models according to the category associated with the operator.

The invention further relates to a computer program including software instructions which, when executed by a computer, implement a monitoring method as defined hereinabove.

### BRIEF DESCRIPTION OF THE DRAWINGS

Such features and advantages of the invention will become clearer upon reading the following description, provided only as a non-limiting example, and with reference to the enclosed drawings, wherein:

FIG. 1 is a schematic representation of an aircraft including a monitoring device according to the invention;

FIG. 2 is a schematic view of control station inside the aircraft shown in FIG. 1;

FIG. 3 is a flow chart of the construction and training method of a model according to the invention; and

FIG. 4 is a flow chart of a monitoring method according to the invention, as used by the monitoring device.

### DETAILED DESCRIPTION OF EMBODIMENTS

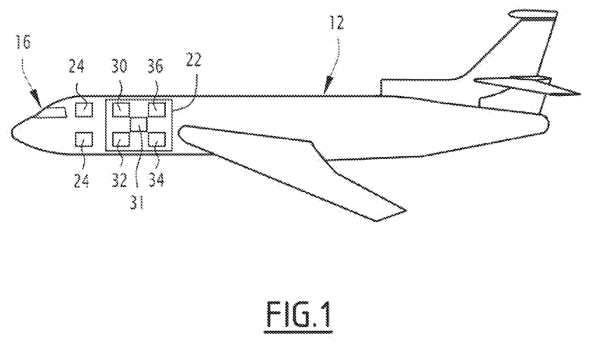

An aircraft 12 is shown in FIG. 1.

Aircraft 12 is typically an airplane, a helicopter, or a drone. In other words, aircraft 12 is a flying machine which may be piloted by an operator 14 via a control station 16. Control station 16 is arranged inside aircraft 12 or at a distance from aircraft 12, in particular in the case of a drone.

Operator 14 is herein a pilot, but the invention applies in a similar manner to any operator of aircraft 12 such as a co-pilot or a radar operator.

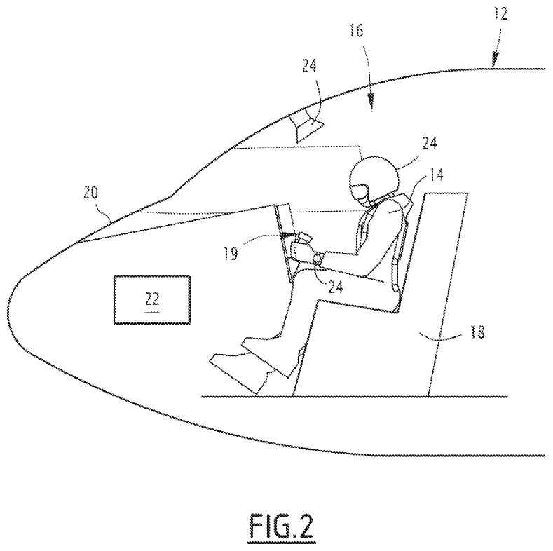

As may be seen in FIG. 2, control station 16 is herein a cockpit of aircraft 12. As may be seen in FIG. 1, control station 16 includes at least one seat 18 for operator 14, a control device 19 suitable for being actuated by operator 14, a windscreen 20 at least partially transparent and separating the inside of the cockpit from the outside environment of aircraft 12, at least one sensor 24, and an electronic monitoring device 22 of a neurophysiological state of operator 14.

Each sensor 24 is configured for measuring at least one piece of information relating to operator 14 and in particular to his/her neurophysiological state, as will be explained in greater detail thereafter.

Each sensor 24 is in particular, a so-called worn sensor or a so-called remote sensor.

A “worn sensor” is a sensor suitable for being in physical contact with operator 14. A person skilled in the art will understand that “in physical contact” means that sensor 24 touches a part of operator 14, possibly with a garment between sensor 24 and the skin of operator 14. Thereby, worn sensor 24 is, e.g., in the form of a watch on the wrist of operator 14, a helmet on the head of operator 14, or a sensor integrated into control device 19 or in seat 18.

A “remote sensor” is a sensor arranged remotely from operator 14 during operational flight conditions. A person skilled in the art will understand that the term “remotely” means that there is an empty space between sensor 24 and operator 14.

In particular, each sensor 24 is chosen from the group consisting of:

a cardiac sensor, in particular an electrocardiograph;

a pulse oximeter, including a photoplethysmography sensor;

a respiration sensor;

an accelerometer;

a scalp electrode, e.g. an electroencephalograph;

a pressure sensor arranged in seat 18 of operator 14;

a pressure sensor arranged in control device 19 suitable for being actuated by operator 14;

a sweating sensor for the operator;

a galvanic skin response sensor;

a camera configured for taking at least one image including at least part of operator 14, in particular eyes for eye tracking;

a microphone;

an infrared sensor for the skin temperature of operator 14;

an internal temperature sensor for operator 14; and

a near-infrared spectroscopy headband (also called “NIRS” headband) using near-infrared light to monitor brain activity.

Electronic monitoring device 22 is configured for monitoring a neurophysiological state of operator 14. The neurophysiological state is related to the nervous system of operator 14. The neurophysiological state is representative of the ability of operator 14 to act for carrying out the tasks required for the safety of aircraft 12, e.g., piloting aircraft 12 or responding to external communications for a pilot.

In particular, the neurophysiological state may be a so-called “nominal” neurophysiological state, corresponding to the expected neurophysiological state of operator 14 during a flight of aircraft 12, i.e., an awake and clear-headed state.

The neurophysiological state may be a so-called “altered” neurophysiological state, corresponding to a neurophysiological state wherein the neurophysiological state of operator 14 impacts his/her ability to act for ensuring the safety of aircraft 12. An altered neurophysiological state is, e.g., a state of stress, fatigue, mental load either too low or too high, either hypo or hyper-vigilance (or tunneling), etc. An altered neurophysiological state may also be a state where operator 14 suffers from at least partial loss of awareness about the outside world, such as, e.g., a state of drowsiness, sleep or fainting. In such neurophysiological state, operator 14 has an altered or non-existent knowledge of his/her environment and cannot react accordingly. Such altered neurophysiological state is problematic during flight of aircraft 12 because operator 14 is not able to carry out the tasks he/she has to perform in a reactive and relevant way.

To this end, monitoring device 22 is configured for determining whether operator 14 is in a nominal neurophysiological state or in an altered neurophysiological state.

In particular, monitoring device 22 includes a receiver module 30, a categorization module 31, a processing module 32, and a detection module 34.

Advantageously, monitoring device 22 further includes a warning module 36.

Receiver module 30 is configured for receiving a datum from at least one sensor 24 on-board aircraft 12, in particular in control station 16.

Categorization module 31 is configured for associating with operator 14, one category from a list of categories predetermined from the datum or data received by receiver module 30.

A category is a class within which elements are arranged according to a number of criteria. In the present case, the predetermined categories may be used for classifying different operators 14 according to a list of predetermined attributes. Thereby, a category may be used for characterizing the variabilities of each operator 14 by associating the operator with a category.

The list of categories is predetermined before the operational phases of flight, in particular with the help of experts in the field.

Categorization module 31 is configured for associating a category with operator 14, according to the attributes relating to operator 14.

In particular, categorization module 31 is configured for associating a category according to at least one so-called individual attribute. An “individual attribute” is a physical or physiological attribute specific to operator 14. Each individual attribute is chosen, in particular, from the group consisting of: gender, age, ethnicity, pilosity, hair length, presence of elements on the skin of the face, such as tattoos, makeup, scars, etc.

In a variant or in addition, categorization module 31 is configured for associating a category according to at least one so-called worn accessory attribute. A “worn accessory attribute” is an attribute relating to a garment or an object worn by operator 14. Each worn accessory attribute is chosen from the group consisting of: wearing glasses, either polarized or non-polarized glasses, lenses, surgical mask, gas mask, headphones, cap.

Processing module 32 is configured for extracting from each datum, at least one parameter representative of the neurophysiological state of operator 14.

A person skilled in the art will understand that categorization module 31 and processing module 32 do not necessarily use the same data coming from the sensors. A sensor, such as a camera, is used by categorization module 31, and another sensor, such as a cardiac sensor, is used by processing module 32. However, in one possible embodiment, modules 31 and 32 use the same data, e.g., the images coming from a camera in control station 16.

A parameter representative of the neurophysiological state is a parameter defined, e.g., by experts in the field and giving information on the neurophysiological state of the operator. A low heart rate, eyes closed over a long period of time, constant pressure exerted, a tilted head position, etc., are, e.g., parameters for determining that the operator is in an altered neurophysiological state.

Advantageously, processing module 32 is configured for extracting from each datum at least one parameter representative of the neurophysiological state of operator 14 according to the category associated with operator 14.

In particular, processing module 32 is configured for adapting which parameter from a list of possible parameters is extracted according to the category of operator 14.

As an example, processing module 32 may adapt a parameter extracted from images according to whether or not operator 14 is wearing glasses. If operator 14 does not wear glasses, processing module 32 extracts blinking of the eyes from the images, whereas if operator 14 does wear glasses, processing module 32 extracts a movement of the head.

In a variant or in addition, processing module 32 is configured for adjusting the way of extracting a parameter according to the category associated with operator 14.

As an example, a threshold for detecting blinking of the eyes of operator 14 is adjusted according to the category of operator 14. Indeed, the detection threshold of an eye blink is not the same between an Asian person and a Caucasian person, because of the average opening of the eyelid or of the characteristics of the epicanthic fold.

Advantageously, processing module 32 is configured for extracting from each datum, at least one parameter representative of the neurophysiological state of operator 14 by performing an extraction of a predetermined characteristic of the associated datum followed by a machine learning method.

As an example, the characteristic is the position of the head of operator 14, extracted from a video taken by a camera. A machine learning method is then implemented for processing the position of the head over time and for inferring therefrom a parameter representative of the neurophysiological state of operator 14.

A machine learning method is used for obtaining a model apt to solve tasks without being explicitly programmed for each of the tasks. Machine learning includes two phases. The first phase consists in defining a model from data present in a learning database, also called observations. The definition of the model consists herein in training the model to recognize a deleterious neurophysiological state. The so-called learning phase is generally carried out prior to the practical use of the model. The second phase corresponds to the use of the model: the model being defined, new data may then be submitted to the model in order to determine the neurophysiological state of operator 14.

In a variant, processing module 32 is configured for extracting from each datum, at least one parameter representative of the neurophysiological state of operator 14 by implementing a deep learning method applied directly to the associated datum.

A deep learning method is a technique based on the model of neural networks, or networks of neurons: tens or even hundreds of layers of neurons are stacked for bringing greater complexity to the model. In particular, a neural network generally consists of a succession of layers, each of which takes the inputs thereof from the outputs of the preceding layer. Each layer consists of a plurality of neurons, taking the inputs thereof from the neurons of the preceding layer. Each synapse between neurons is associated with a synaptic weight, so that the inputs received by a neuron are multiplied by the weight and then added by the neuron. The neural network is optimized by adjusting the different synaptic weights during training, according to data in the learning database. The neural network thereby optimized then becomes the model. A new set of data may then be given at the input of the neural network, which then supplies the result of the task for which the neural network has been trained.

In a variant, processing module 32 is configured for extracting from each datum, at least one parameter representative of the neurophysiological state of operator 14 by implementing a pre-determined model applied to the associated datum.

The predetermined model is, e.g., a physical model including a set of rules predetermined by an expert in the field.

In an advantageous embodiment, processing module 32 is configured for extracting from each datum, at least one parameter representative of the neurophysiological state of operator 14, by implementing an algorithm chosen according to the category of operator 14 from the group consisting of:

an extraction of a predetermined characteristic of the associated datum followed by a machine learning method;

a deep learning method applied directly to the associated datum; and

a predetermined modeling applied to the associated datum.

As an example, for a category wherein the associated deep learning method has been trained on a large number of data and with good performance results, such method will be preferred. On the other hand, for a category with little experience data and where machine learning methods are less efficient, a predetermined model is preferred so as to avoid false positives.

Detection module 34 is configured for receiving the category associated with operator 14 determined by categorization module 31 and the representative parameter or parameters determined by processing module 32.

Detection module 34 is further configured for applying a model coming from a machine learning method, for determining, according to the representative parameters, whether operator 14 is in a nominal neurophysiological state or in an altered neurophysiological state.

The automatic learning method used by detection module 34 is different from, or possibly similar to, the method used by processing module 32.

The model is chosen from a list of predetermined models according to the category associated with operator 14.

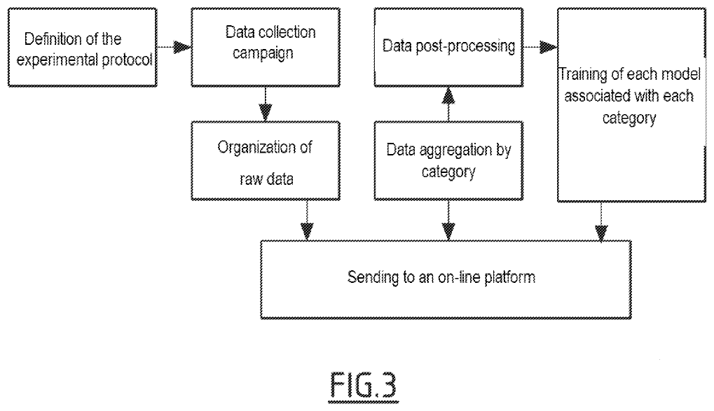

FIG. 3 shows a flow chart of the process of the construction and training of such predetermined models.

Initially, the experimental protocol is set up. It is a question of defining for the neurophysiological state that one wants to monitor:

how to generate such state in subjects;

relevant sensors to monitor the neurophysiological state;

the means of collecting the “ground truth”, also called the label, i.e., what state the subject is in (e.g., normal fatigue or extreme fatigue);

the number of subjects required for making the model sufficiently representative, as well as the inter-individual variabilities or in terms of the objects worn to be addressed;

the collection environment (laboratory, flight, etc.) and the representativeness thereof with respect to the final use of the system; and

potential biases related to the experimentation.

During the protocol definition phase, the method includes identifying different combinations of variabilities to be monitored. In particular, it is investigated during such stage, which parameters are significant and relevant for characterizing the neurophysiological state of operator 14 according to each category, in particular with the help of experts in the field.

Combinations of variabilities may depend only on one parameter, such as making a model for men and one for women, but could also contain a large number of parameters, such as a model for Asian men wearing glasses. The number of models to be created depends on the number of combinations.

As a result, data is collected during campaigns. The result therefrom is a database of labeled physiological data, i.e., associated with a precise physiological state.

Since the data are raw, the data are processed. The quality of the data is then estimated according to supplementary criteria. The quality of face detection, e.g., may be determined using a face tracking algorithm. Data sampling, the presence of artifacts or sensor noise are also checked. It is also possible to remove the trend of the signals, i.e., low frequency elements not relevant for monitoring.

At the end of the data collections, the data are grouped into the identified combinations of attributes so as to form the different combinations. A model is then developed for each category, i.e. each combination.

Thus, after collecting training data on a plurality of operators, the different operators are, e.g., classified according to the variability “gender”, i.e. male or female, or e.g. according to the variability “ethnic origin”, i.e. Caucasian, African, Asian, Mediterranean, etc. The categories may also be formed by making combinations of two or more variabilities for each operator, e.g., male and Asian, or female and Australian, and the model trained only on data from Asian males or Australian females in this example.

At each operation of the process, elements are advantageously transmitted to an on-line platform authorized to host GPDR (General Data Protection Regulation) and HDS (Health Data Hosting) data.

Warning module 36 is configured for issuing a warning signal when detection module 34 determines that the operator is in an altered neurophysiological state.

The warning signal is, e.g., a sound signal emitted in control station 16 with the aim of returning operator 14 to a nominal neurophysiological state.

In a variant or in addition, the warning signal is, e.g., a signal sent to a control system of aircraft 12 in order to switch to automatic mode and so that the tasks to be performed by operator 14 are carried out autonomously without intervention of operator 14. In particular, when operator 14 is a pilot, aircraft 12 switches to autopilot.

In a variant or in addition, the warning signal is, e.g., a communication signal to a control device external to aircraft 12, such as a control tower.

In the example shown in FIG. 1, electronic monitoring device 22 includes an information processing unit including, e.g., a memory and a processor associated with the memory. Receiver module 30, categorization module 31, processing module 32, detection module 34, and warning module 36 are each implemented in the form of a software program, or a software brick, which may be run by the processor. The memory is then apt to store a receiver software, a categorization software, a processing software, a detection software, and, as an optional addition, a warning software. The processor is then apt to run each of the software programs.

In a variant (not shown), receiver module 30, categorization module 31, processing module 32, detection module 34 and, as an optional addition, warning module 36 are each produced in the form of a programmable logic component, such as an FPGA (Field Programmable Gate Array), or further in the form of a dedicated integrated circuit, such as an ASIC (Application Specific Integrated Circuit).

When electronic tool 22 is produced in the form of one or a plurality of software programs, i.e., in the form of a computer program, the computer program is further apt of being recorded on a computer-readable medium (not shown). The computer-readable medium is, e.g., a medium apt to store the electronic instructions and to be coupled to a bus of a computer system. As an example, the computer-readable medium is an optical disk, a magneto-optical disk, a ROM memory, a RAM memory, any type of non-volatile memory (e.g., EPROM, EEPROM, FLASH, NVRAM), a magnetic card, or an optical card. A computer program containing software instructions is then stored on the readable medium.

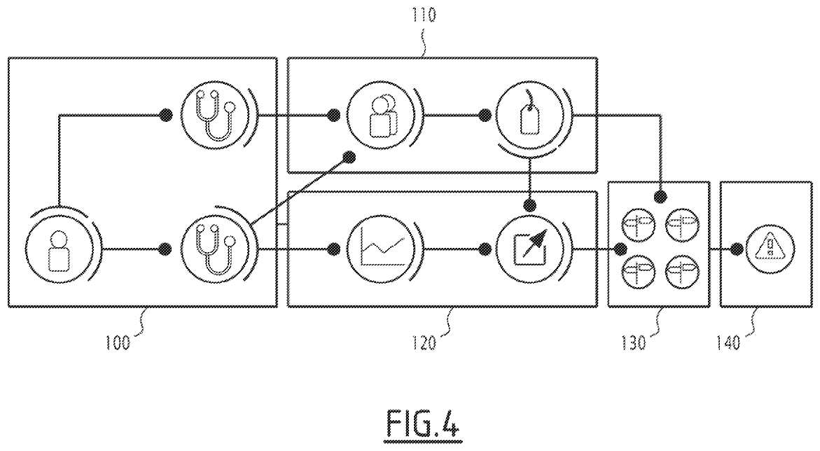

The operation of electronic monitoring device 22 according to the invention will now be explained using FIG. 4, which shows a flow chart of the method according to the invention, for monitoring a neurophysiological state of operator 14 in control station 16 of aircraft 12.

Initially, aircraft 12 is in an operational flight situation, flying, e.g., to an airport.

At least one operator 14 is present in control station 16 of aircraft 12. Operator 14 is, e.g., a pilot, as shown herein.

As an example, the pilot is, e.g., an Asian man wearing glasses.

The method includes an initial operation 100 of reception by receiver module 30 of a datum from at least one sensor 24 on-board aircraft 12.

Each sensor 24 is configured for measuring at least one piece of information relating to the pilot.

Herein, as illustrated in FIG. 2, receiver module 30 receives, e.g., images of the face of the pilot, coming from a camera arranged in control station 16 and cardiac data of the pilot from a watch on the pilot's wrist.

Then, the method includes an operation 110 of associating with operator 14, one category from a list of categories predetermined from the datum or data received by categorization module 31.

In the preceding example, categorization module 31 determines from the images that the gender of the pilot is “male”, that the “ethnic origin” of the pilot is “Asian”, and that the object worn on the face is “glasses”. From such attributes, categorization module 31 associates the pilot with the category “Asian man with glasses”, which forms part of the predetermined categories available for categorization module 31.

Then, the method includes an operation 120 of extracting each datum of at least one parameter representative of the neurophysiological state of operator 14.

In the preceding example, processing module 32 extracts cardiac data from the pilot, in particular the heart rate, and compares the heart rate with a rest threshold value below which there is a suspicion of an altered neurophysiological state of the pilot. In a variant or in addition, processing module 32 extracts movement of the pilot's head from the images.

Advantageously, processing module 32 extracts from each datum, at least one parameter representative of the neurophysiological state of operator 14 according to the category associated with operator 14.

In the preceding example, processing module 32 prevents extraction of the blinking frequency of the eyes of the operator, because such extraction is less reliable because of the presence of the glasses.

The method then includes an operation 130 of receiving, by detection module 34, the category associated with operator 14 and the representative parameter or parameters, and applying a model derived from an automatic learning method, for determining, according to the representative parameters, whether operator 14 is in a nominal neurophysiological state or in an altered neurophysiological state. The model is chosen from a list of predetermined models according to the category associated with operator 14.

Still in the previous example, detection module 34 uses the model associated with the category “Asian man with glasses”. Such model was specially trained on training data relating to the category of pilot, and is thus particularly effective in determining the neurophysiological state of the pilot. Herein, from the heart rate and/or the movements of the pilot's head, detection module 34 determines whether the pilot is in a nominal neurophysiological state or in an altered state. If detection module 34, e.g., receives a low heart rate and/or a swinging of the head laterally, the model may determine, if appropriate, an altered neurophysiological state of the pilot.

Advantageously, the method includes an operation 140 of emitting a warning signal when detection module 34 determines that operator 14 is in an altered neurophysiological state.

In this way, it may be understood that the present invention has a certain number of advantages.

Indeed, the device according to the invention provides a high-performance monitoring of the neurophysiological state of operator 14 while coping with the variabilities of the monitored operators. The prior categorization of operator 14 ensures sufficient coverage of the monitoring, while offering higher performance on each specific model. Indeed, monitoring of the operator 14 is carried out by a model associated with each category, trained specifically on data relating to the category. Each specific model thus provides a higher performance than with a unique model which would seek to cover all possible variabilities of the operators.

The invention may thus be used for a better detection of altered neurophysiological states of the operators and reduces false positives. Thus, the invention improves the safety of aircraft 12.

1. An electronic monitoring device for monitoring a neurophysiological state of an operator in a control station of an aircraft, the monitoring device comprising:
a receiver module receiving data from at least one sensor on-board the aircraft, each sensor measuring at least one piece of information relating to the operator;
a categorization module associating the operator with one category from a list of predetermined categories, from the received datum or data;
a processing module extracting from each datum, at least one parameter representative of the neurophysiological state of the operator;
a detection module receiving the category associated with the operator and the representative parameter(s), and applying a model derived from a machine learning method, for determining, according to the representative parameter(s), whether the operator is in a nominal neurophysiological state or in an altered neurophysiological state, the model being chosen from a list of predetermined models according to the category associated with the operator.

2. The monitoring device according to claim 1, further comprising a warning module issuing a warning signal when said detection module determines that the operator is in an altered neurophysiological state.

3. The monitoring device according to claim 1, wherein each sensor is chosen from the group consisting of:
a cardiac sensor;
a pulse oximeter;
a respiration sensor;
an accelerometer;
a scalp electrode;
a pressure sensor arranged in a seat of the operator;
a pressure sensor arranged in a control device and actuated by the operator;
a sweating sensor for the operator;
a galvanic skin response sensor;
a camera taking at least one image including at least part of the operator;
a microphone;
an infrared sensor for the skin temperature of the operator;
an internal temperature sensor for the operator;
a near-infrared spectroscopy headband.

4. The monitoring device according to claim 3, wherein the cardiac sensor is an electrocardiograph.

5. The monitoring device according to claim 3, wherein the pulse oximeter is a photoplethysmography sensor.

6. The monitoring device according to claim 3, wherein the scalp electrode is an electroencephalograph.

7. The monitoring device according to claim 1, wherein said categorization module associates a category according to at least one individual attribute chosen from the group consisting of: gender, age, ethnicity, pilosity, hair length, and presence of elements on the skin of the face.

8. The monitoring device according to claim 1, wherein said categorization module associates a category according to at least one attribute called a worn accessory chosen from the group consisting of:
wearing glasses, polarized or non-polarized glasses, lenses, surgical mask, gas mask, headphone, and cap.

9. The monitoring device according to claim 1, wherein said processing module extracts from each datum, at least one parameter representative of the neurophysiological state of the operator according to the category associated with the operator.

10. The monitoring device according to claim 1, wherein said processing module extracts from each datum, at least one parameter representative of the neurophysiological state of the operator by implementing, for each datum, an algorithm chosen from the group consisting of:
an extraction of a predetermined characteristic of the associated datum followed by a machine learning method;
a deep learning method applied directly to the associated datum; and
a predetermined modeling applied to the associated datum.

11. The monitoring device according to claim 10, wherein the algorithm used is chosen according to the category of the operator.

12. A method for monitoring a neurophysiological state of an operator in a control station of an aircraft, the method comprising:
receiving data from at least one sensor on-board the aircraft, each sensor measuring at least one piece of information relating to the operator;
associating with the operator one category from a list of categories predetermined from the received datum or data;
extracting from each datum, at least one parameter representative of the neurophysiological state of the operator; and
receiving the category associated with the operator and the representative parameter(s) and applying a model derived from a machine learning method, for determining, according to the representative parameters, whether the operator is in a nominal neurophysiological state or in an altered neurophysiological state, the model being chosen from a list of predetermined models according to the category associated with the operator.

13. A computer program including software instructions which, when executed by a computer, cause the computer to implement the method according to claim 12.
